(19) United States
(12) Patent Application Publication
(10) Pub. No.: US 2024/0032874 A1
(43) Pub. Date: Feb. 1, 2024

(54) REAL-TIME MONITORING AND EARLY WARNING SYSTEM FOR BLOOD OXYGEN AND HEART RATE

(71) Applicant: NATIONAL TAIWAN UNIVERSITY HOSPITAL, Taipei (TW)

(72) Inventor: CHIEN-CHANG LEE, Taipei (TW)

(21) Appl. No.: 18/360,770

(22) Filed: Jul. 27, 2023

Related U.S. Application Data
(60) Provisional application No. 63/369,518, filed on Jul. 27, 2022.

(51) Int. Cl. A61B 5/00; A61B 5/0205; G16H 50/20; G16H 50/70; G16H 50/30

(52) U.S. Cl. CPC A61B 5/7267; A61B 5/0205; A61B 5/7275; G16H 50/20; G16H 50/70; G16H 50/30; A61B 5/024

(57) ABSTRACT

The invention is related to a real-time platform for blood oxygen and heart rate monitoring based on the Internet of Things (IoT), which may collect real-time blood oxygen and heart rate information of patients for analysis, and use it to predict the probability of a patient's health emergency, so as to facilitate medical personnel to quickly grasp the situation and quickly intervene in treatment. On the other hand, the real-time information collected from patients can also be used as statistical and training data for analysis and warning to further optimize the early warning capability of the platform.

### RELATED APPLICATION

This application claims the benefit of the U.S. Provisional Application No. 63/369,518 filed on Jul. 27, 2022, titled “ IOT-BASED REAL-TIME MONITORING AND EARLY WARNING SYSTEM FOR BLOOD OXYGEN AND HEART RATE,” which is incorporated herein by reference at its entirety.

### BACKGROUND OF THE INVENTION

### Field of the Invention

The present invention relates to a real-time blood oxygen and heart rate monitoring and early warning platform, combined with the Internet of Things and machine learning.

### Description of Related Art

Coronavirus disease 2019 (COVID-19) is an infectious disease caused by Severe Acute Respiratory Syndrome Coronavirus Type 2 (SARS-CoV-2). Until June 2022, there were over 500 million confirmed cases and over 6 million deaths worldwide since the first case detected in 2019. It is one of the deadliest epidemics in human history.

Because of the limited medical resources compared to the number of infected cases, many patients (especially for those who show no/mild symptoms) are not hospitalized or even not diagnosed. Consequently, many reported deaths were those who died in the community before being diagnosed, or those who diagnosed but deteriorated rapidly when isolated at home or in quarantine. In the above-mentioned cases, many patients experienced silent hypoxia but did not realize it until too late for treatment.

Hypoxia is a condition in which body tissues contain little oxygen, often caused by hypoxemia, which is a lower than normal level of oxygen in the blood. The normal arterial partial pressure of oxygen (PaO₂) is about 75-100 millimeters of mercury (mm Hg). A value lower than 60 mm Hg means hypoxia, and oxygen therapy is required. Hypoxia can seriously affect the function of all organs in the body, especially the brain and heart.

Silent hypoxia refers to the situation where the body already has low blood oxygen while the patient does not feel dyspnea. Compared with other respiratory infectious diseases, COVID-19 is particularly prone to silent hypoxia. According to statistics, ⅓ of patients with hypoxia caused by COVID-19 do not feel particularly difficult or short of breath. However, the actual measurement of blood oxygen shows that the patient's body already has hypoxic conditions. Therefore, it is easy for those patients to delay medical treatment, and more likely to result in sudden death. Under the condition of silent hypoxia, the patient does not feel discomfort or pain when the body is hypoxic, and does not realize that the condition is serious. As a result, some patients' condition deteriorates rapidly and cannot be rescued in time.

In the case of rapid deterioration, sudden death caused by cardiac arrest (CA) is the most unexpected and fatal. However, studies have found that minor changes on various physiological data of patients emerged a few hours before the occurrence of cardiac arrest, but these changes are difficult to interpret manually.

In the past, some attempts to predict the risk of cardiac arrest used rule-based judgments, such as judging that a certain physiological signal (e.g. blood pressure, heart rate, respiratory rate, etc.) is greater than or less than a certain threshold is considered a high risk. However, when there are few rules, this type of method tends to make predictions inaccurate due to individual differences; and when there are many rules, the prediction will only be able to be carried out in large hospitals but not ordinary housing due to measurement of too many physiological signals. Considering that many patients with mild symptoms of COVID-19 are self-monitoring at home, it is difficult for the above prediction methods to take care of these patients.

In order to solve the aforementioned problems, there is a need to develop a real-time physiological data monitoring and early warning platform, which can automatically collect a small number of important physiological data of patients, and can accurately predict the health status of patients (such as whether there is a risk of sudden death) with only those few physiological data, and then provide the analysis results to the patient himself and remote medical staff.

### SUMMARY OF THE INVENTION

The present invention provides an Internet of Things (IoT)-based platform that can automatically collect real-time blood oxygen and heart rate information of patients and perform analysis and early warning. The platform can use only blood oxygen and heart rate of the patient, two physiological data which can be easily measured by ordinary household pulse oximeters, to accurately predict whether the patient is at risk of cardiac arrest. In addition, the real-time information collected from the patient will also be used as statistical and training data for analysis and early warning to further optimize the early warning capabilities of the platform.

In one aspect, the current invention is related to a machine learning model training method to train a sudden death prediction model for predicting the probability of sudden death, comprising using continuous physiological monitoring data and survival results of patients in a database for model training, wherein the physiological monitoring data consist essentially of heart rate (HR) and blood oxygen (SpO₂) data, and the survival results comprise categories of the survival status of the patients.

In one embodiment, the physiological monitoring data comprise continuous 24-hour physiological data of patients.

In one embodiment, the categories of the survival status comprise a category representing patient alive and a category representing patient death.

In one embodiment, the model training method comprises performing synthetic minority oversampling technique (SMOTE) for categories with smaller data amount for repeated sampling, so that the amount of data of different categories are approximately equal.

In one embodiment, the machine learning model is trained with a long short-term memory recurrent neural network (LSTM-RNN). In a preferred embodiment, the LSTM-RNN employs two LSTM layers plus two fully connected layers after expansion of the LSTM layers, and then applies a Softmax layer to the last layer for training.

In one embodiment, the machine learning model is trained by temporal convolutional network (TCN).

Another aspect of the current invention is related to a trained sudden death prediction model for predicting the probability of sudden death of a subject, the sudden death prediction model can use continuous physiological monitoring data of the subject to predict the health status of the subject, wherein the sudden death prediction model is a machine learning model, and the physiological monitoring data consist essentially of heart rate (HR) and blood oxygen (SpO₂) data.

In one embodiment, the health status is a predicted survival status of the subject. In another embodiment, the health status is the probability of sudden death of the subject. In some embodiments, the model can predict sudden death of the subject 1 hour, 3 hours, 6 hours or even 9 hours before occurrence of cardiac arrest. In a specific embodiment, the model can predict sudden death of the subject 6 hours before occurrence of cardiac arrest.

Yet another aspect of the current invention is related to a real-time monitoring system for monitoring and warning the risk of sudden death of patients, comprising at least one sensing module capable of real-time measurement and transmission of physiological data, a server, and a plurality of remote devices, wherein: the sensing module is connected to the server, and the physiological data measured by the sensing module can be transmitted to the server in real time, the physiological data comprise at least heart rate (HR) and blood oxygen (SpO₂) data of a subject; the server comprises a prediction platform comprising a trained sudden death prediction model, and the trained sudden death prediction model is capable of predicting the health status of the subject only relying on the heart rate (HR) and blood oxygen (SpO₂) data of the subject; and the server is connected with the remote devices, and can transmit the real-time physiological data measured by the sensing module and the health status predicted by the prediction platform to the remote devices with access authority.

In one embodiment, the sensing module is a household pulse oximeter capable of collecting heart rate (HR) and blood oxygen (SpO₂) data.

In one embodiment, the server comprises a SQL database system.

In one embodiment, the server comprises a Power BI analysis reporting system.

It should be understood that the above general descriptions and the following detailed descriptions are only for demonstrative and explanatory purposes, instead of limiting the scope of the present invention.

### BRIEF DESCRIPTION OF THE DRAWINGS

The above general descriptions and the following detailed descriptions of the present invention can be better understood with the figures. To illustrate the present invention, the preferred embodiments are provided in the figures. However, it should be understood that the present invention is not limited to the embodiments disclosed in the figures.

FIG. 1 shows the system architecture diagram of the present invention.

FIG. 2 is the network architecture of LSTM training in this invention.

FIG. 3 is the network architecture of TCN training in this invention.

FIG. 4 is the receiver operating characteristic curve (ROC curve) showing the prediction performance to the test data for the prediction system trained with LSTM in the present invention. The area under the curve (AUC) is 96.7%.

FIG. 5 shows the ROC curve of the prediction performance to the overall data for the prediction system trained with LSTM in the present invention. The area under the curve (AUC) is 96.7%.

FIG. 6 shows the ROC curve of the prediction performance to the test data for the prediction system trained with TCN in the present invention. The area under the curve (AUC) is 96.8%.

FIG. 7 shows the ROC curve of the prediction performance to the overall data for the prediction system trained with TCN in the present invention. The area under the curve (AUC) is 96.8%.

FIG. 8 shows that the ROC curve of the prediction performance to the test data for the six-parameter (heart rate, respiratory rate, systolic blood pressure, diastolic blood pressure, average arterial pressure, and blood oxygen) prediction system trained with TCN. The area under the curve (AUC) is 99.5%.

FIG. 9 shows that the ROC curve of the prediction performance to the overall data for the six-parameter (heart rate, respiratory rate, systolic blood pressure, diastolic blood pressure, average arterial pressure, and blood oxygen) prediction system trained with TCN. The area under the curve (AUC) is 99.5%.

FIG. 10 shows the sensitivity data for the prediction of the two-parameter (HR and SpO₂) model from 12 hours to 1 hour before CA occurrence.

FIG. 11 shows the specificity data for the prediction of the two-parameter (HR and SpO₂) model from 12 hours to 1 hour before CA occurrence.

FIG. 12 shows the AUC for the prediction of the two-parameter (HR and SpO₂) model from 12 hours to 1 hour before CA occurrence.

FIG. 13 shows the sensitivity data for predictions of MEWS, CART and NEWS from 12 hours to 1 hour before CA occurrence.

### DETAILED DESCRIPTION OF PREFERRED EMBODIMENTS

The following examples are referred to clearly explain the technical content, features and effects of this invention. Through the explanation of specific embodiments, a skilled artisan can further understand the technical means and effects adopted in the present invention to achieve the aforementioned purpose of the invention. In addition, the technology disclosed in this specification can be understood and implemented by a skilled artisan. Any changes or improvements consistent with the inventive concept are within the scope of the claims.

All the technical and scientific terms described in this specification and the claims, unless otherwise defined, are all with the definitions known by a person with ordinarily skilled in the art of this invention. A singular article “one”, “a”, “an”, “the”, or its approximate term, unless otherwise specified, can refer to more than one subject. The terms “or”, “and” used in the specification, unless otherwise specified, all refer to “and/or”. In addition, the terms “comprise” and “include” are open-ended terms which are not limiting. The aforementioned definition only describes the directed definition of the terms and should not be interpreted as a limitation to the claimed invention. Unless otherwise specified, the materials used in the present invention are all commercially available.

The term “sensing module” used herein refers to a device that can collect physiological data (e.g. blood oxygen and heart rate) of an object (e.g. a patient in a hospital) and transmit the collected physiological data to a module of a remote server. The above-mentioned transmission methods include but not limited to Bluetooth mesh networking, Li-Fi, Near-field communication (NFC), radio-frequency identification (RFID), Wi-Fi, ZigBee, Z-Wave, mobile network, low-power wide-area network, (LPWAN), and wired network, and may also combine a variety of technologies, such as Bluetooth transmission to the local gateway, and then transmission through the wireless network from the gateway to the remote server.

The term “server” used herein refers to a computer software module that manages resources and provides services, or a computer that executes the above software. Its functions include but not limited to data storage, database services, and execution of applications (such as data analysis and processing, alerting, and etc.), and may combine multiple functions, such as combining SQL database system and Power BI analysis and reporting system. The server may receive and store the physiological data measured by the sensing module, and send the result to the remote accessing module (such as a remote device) for use after internal analysis.

In general, the present invention provides a device which collects continuous blood oxygen and heart rate data to predict the risk of sudden death (e.g. risk of cardiac arrest). In this device there is a computing unit configured to take only blood oxygen (SpO₂) and heart rate (HR) as input to produce an output of predicted risk of cardiac arrest. The computing unit may be implemented remotely (e.g. on the cloud or a remote server) to analyze incoming blood oxygen and heart rate data from a conventional monitoring device such as an oximeter. Alternatively, the computing unit may also be integrated into a monitoring device (e.g. an oximeter) which measures and collects blood oxygen and heart rate as an embedded system to predict cardiac arrest. The monitoring device may be any sensor capable of providing continuous real-time blood oxygen and heart rate monitoring data of a subject for the computing unit's analysis. The algorithm in the computing unit may be a machine learning-based algorithm, or it may also be a rule-based algorithm, as long as it is capable of predicting the risk of cardiac arrest of a subject with blood oxygen (SpO₂) and heart rate (HR) data only.

The present invention also provides a method of predicting cardiac arrest, comprising measuring continuous blood oxygen and heart rate monitoring data and computing a risk of sudden death (e.g. risk of cardiac arrest) using the measured blood oxygen and heart rate monitoring data, wherein the risk is computed by an algorithm capable of predicting cardiac arrest using only blood oxygen (SpO₂) and heart rate (HR) data. The algorithm may be a machine learning-based algorithm, or it may also be a rule-based algorithm.

Referring to FIG. 1, in a preferred embodiment, the present invention provides a real-time monitoring and early warning platform 100 of blood oxygen and heart rate based on the Internet of Things (IoT), comprising at least one sensing module 110 that can measure blood oxygen (SpO₂) and heart rate in real time, a server 120, and a plurality of remote devices 130. The sensing module 110 is connected to the server 120, and the physiological data (blood oxygen and respiratory rate) measured by the sensing module 110 can be transmitted to the server 120 in real time; the server 120 comprises a prediction platform, and uses the real-time physiological data sent by the sensing module 110 to predict the health status of the patient. In addition, the server 120 may also transmit the real-time physiological data sent by the sensing module 110 and the health status predicted by the prediction platform to the remote devices 130 with access rights, thereby facilitates remote diagnosis and necessary treatment by medical personnel, or enables patients to understand their own physical conditions.

### Example 1

### Establishing AI Prediction Model

In this embodiment, the prediction platform of the server 120 uses machine learning to train artificial intelligence (AI) to judge the risk of a patient having an in-hospital cardiac arrest (IHCA). Physiological monitoring data of 23,457 surviving and 3,922 dead patients obtained from public databases in the United States are used as the data for training, verification and testing in the present invention. Firstly, because the dataset in the database is imbalanced data, a total of 23,457 samples were resampled using the synthetic minority oversampling technique (SMOTE) for the death category. After that, 70% of the 23,457 survival data and 23,457 SMOTE sampled death data were used as training data (a total of 32,840 samples), 15% were used as verification data (a total of 7,037 samples), and 15% were used as test data (a total of 7,037 samples). Each training data contains 24 consecutive hours of heart rate (HR) and blood oxygen (SpO₂) measurements. Each of the above training data originally contained 24 data points, and a new set of data was added between each data point by using the interpolation method, expanding the data to a total of 47 data points. The 47 data points and the survival status of the patient (0 means survival; 1 and 2 means death) constitute a complete training data set.

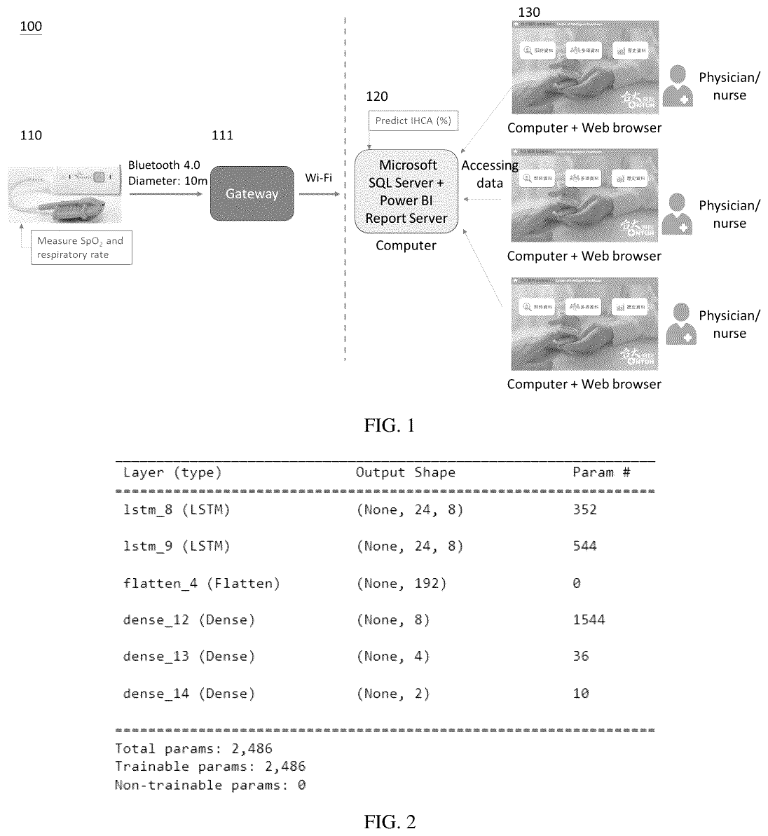

The above training data is trained with long short-term memory (LSTM) recurrent neural network (RNN). The training model employed two LSTM layers plus two fully connected layers after expansion of the LSTM layers, and then applied a Softmax layer to the last layer for training, with a total of 2,486 weights for training, and the performance of the model is monitored with the verification data, as shown in FIG. 2. The method of training optimization adopted the adaptive moment estimation (Adam) optimizer with the initial learning rate of 0.001. After the model was continuously training for 5 epochs, the learning rate decreased exponentially at a rate of 0.9 each time if the monitored numerical performance was not improve, until the value of early stopping reached 20 to stop the training. The process was optimized for at most 500 rounds. The program was executed by a Jupyter Notebook on an A100 GPU while performing the above training.

### Example 2

### Establishing AI Prediction Models with Different Algorithms

The AI prediction model in Example 1 is trained with the algorithm of long-short-term memory recurrent neural network (LSTM). LSTM improves some of the problems of the previous RNN (such as problems of memory design), and it consists of four units: an input gate, an output gate, a memory cell and a forget gate, the control of those gates is also a learnable parameter of the neural network. In addition to LSTM, the temporal convolution network (TCN) algorithm is also used for model training. TCN can handle time series recognition, and the model is mainly composed of fully connected layers in addition to two convolutional layer structures combining causal convolutional layers (causal network) and dilated convolutional layers (dilated network). In the causal convolution we defined that the output value of each layer is only affected by past data, not future data. In addition, the fully connected layer ensures that the output dimension is consistent with the input dimension. Each hidden layer of the expansion convolution is consistent with the input size, and the higher of the causal convolution layer is with larger convolution window (meaning more holes). In addition to increasing the receptive field, this can also reduce the calculation.

In this example, the training was performed with TCN algorithm using the same training data as in Example 1, and the trained model is shown in FIG. 3.

### Example 3

### Prediction Efficacies of the AI Prediction Model

The AI trained in Example 1 and Example 2 were tested for their effectiveness in predicting the risk of sudden death of patients with real patient data, wherein the LSTM model predictive effectiveness is as follows:

The following table is the prediction result using the test data:

| | Predicted no CA | Predicted CA |
|---|---|---|
| Actually no CA | 3,397 | 184 |
| CA actually occurred | 417 | 3,040 |

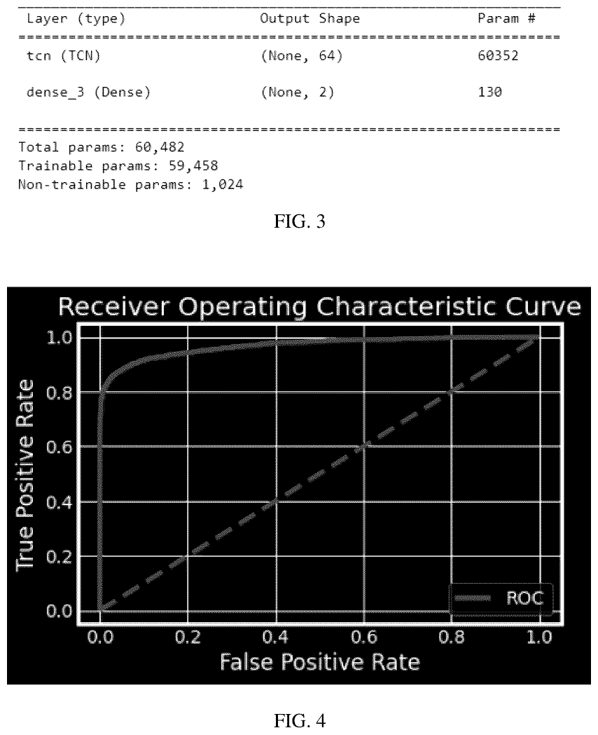

The accuracy is 0.915, the area under the curve (AUC) is 0.967, the sensitivity is 0.879, and the specificity is 0.949. The receiver operating characteristic curve (ROC curve) for the predictive performance of the system is shown in FIG. 4.

The following table is the prediction result using the overall data:

| | Predicted no CA | Predicted CA |
|---|---|---|
| Actually no CA | 22,252 | 1,205 |
| CA actually occurred | 1,039 | 7,720 |

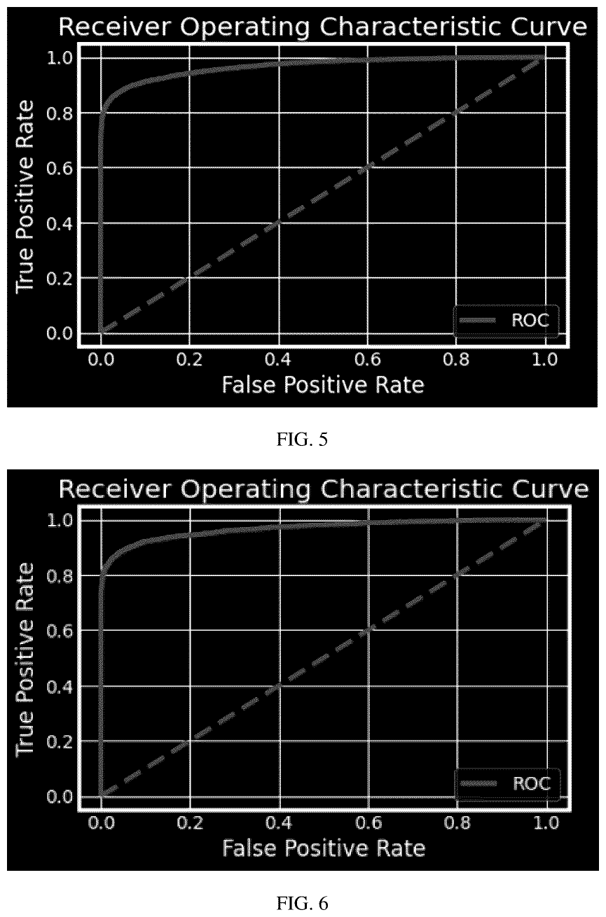

The accuracy is 0.93, the area under the curve (AUC) is 0.967, the sensitivity is 0.881, and the specificity is 0.949. The receiver operating characteristic curve (ROC curve) for the predictive performance of the system is shown in FIG. 5. The above results show that the LSTM model has good predictive power.

On the other hand, the prediction efficacy of the TCN model is as follows:

The following table is the prediction result using the test data:

| | Predicted no CA | Predicted CA |
|---|---|---|
| Actually no CA | 2,893 | 588 |
| CA actually occurred | 218 | 3,339 |

The accuracy is 0.885, the area under the curve (AUC) is 0.968, the sensitivity is 0.939, and the specificity is 0.831. The receiver operating characteristic curve (ROC curve) for the predictive performance of the system is shown in FIG. 6.

The following table is the prediction result using the overall data:

| | Predicted no CA | Predicted CA |
|---|---|---|
| Actually no CA | 19,485 | 3,972 |
| CA actually occurred | 520 | 8,239 |

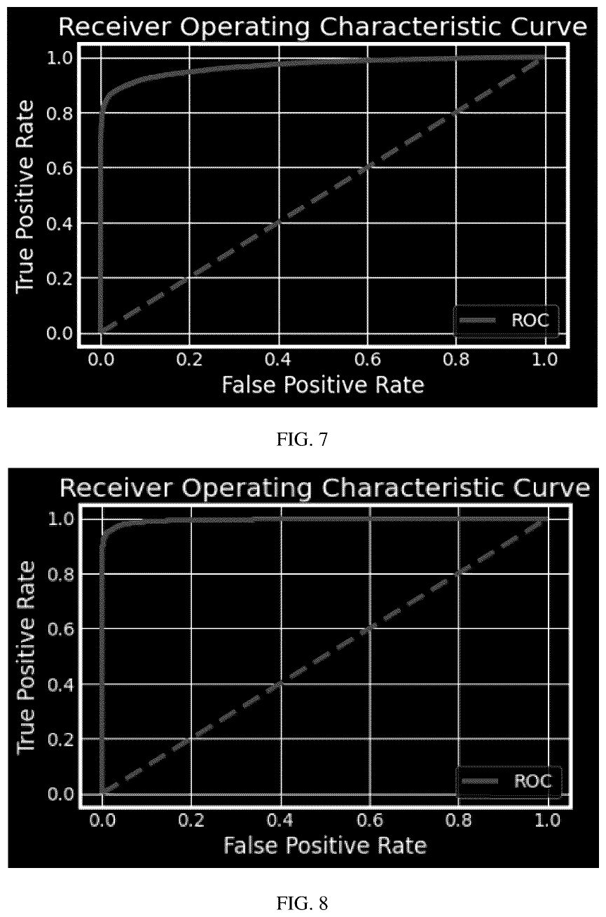

The accuracy (Accuracy) is 0.861, the area under the curve (AUC) is 0.968, the sensitivity is 0.941, and the specificity is 0.831. The receiver operating characteristic curve (ROC curve) for the predictive performance of the system is shown in FIG. 7. The above results show that the TCN model has good predictive power.

From the above results, it can be concluded that good prediction results can be obtained by using both LSTM and TCN. It can be inferred that the monitoring system in the present invention acquired the ability to predict sudden death mainly because of the selection of training data, rather than specific machine learning algorithms. Therefore, this training method should be able to be extended to various machine learning algorithms.

### Example 4

### Comparison to Monitoring and Early Warning Systems with Multiple Physiological Data

The monitoring and early warning system of the present invention is also compared with the traditional monitoring and early warning system that monitors multiple physiological data. The database data from the same source as described above was used to collect 24-hour measurements of heart rate (HR), respiratory rate (RR), systolic blood pressure (SBP), diastolic blood pressure (DBP), mean arterial pressure (MAP) and blood oxygen (SpO₂) of the patients, as well as the survival status of the patient. The training was performed with TCN algorithm. The test results after training are as follows:

The prediction result using the test data:

| | Predicted no CA | Predicted CA |
|---|---|---|
| Actually no CA | 3,283 | 166 |
| CA actually occurred | 82 | 3,507 |

The accuracy is 0.965, the area under the curve (AUC) is 0.995, the sensitivity is 0.977, and the specificity is 0.952. The receiver operating characteristic curve (ROC curve) for the predictive performance of the system is shown in FIG. 8.

The prediction result using the overall data:

| | Predicted no CA | Predicted CA |
|---|---|---|
| Actually no CA | 22,390 | 1,067 |
| CA actually occurred | 189 | 8,570 |

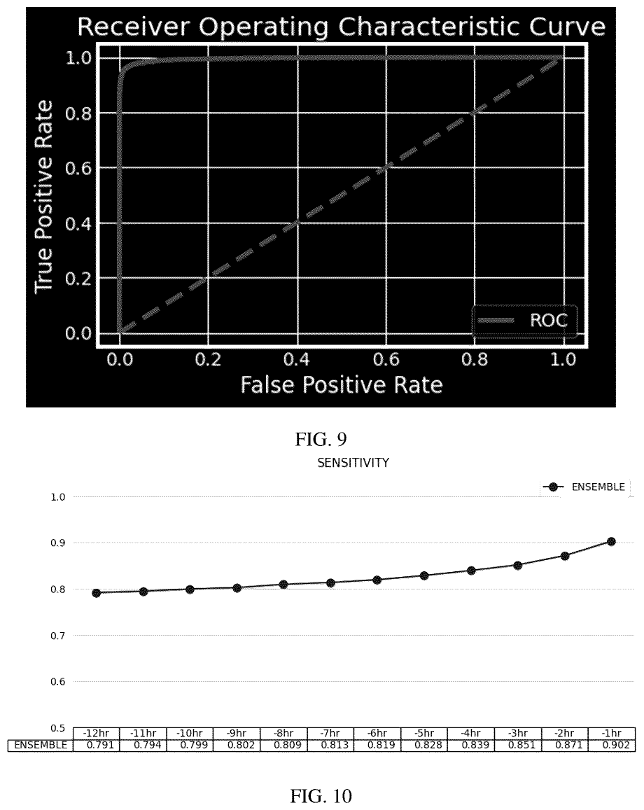

The accuracy is 0.961, the area under the curve (AUC) is 0.995, the sensitivity is 0.978, and the specificity is 0.955. The receiver operating characteristic curve (ROC Curve) for the predictive performance of the system is shown in FIG. 9.

From the above results, it can be seen that the use of heart rate (HR) and blood oxygen (SpO₂) to predict cardiac arrest in the present invention, compared with those using six physiological signals for prediction, may also achieve good prediction performance. And because in the present invention only heart rate (HR) and blood oxygen (SpO₂) data need to be captured for prediction, it is more suitable for integration into ordinary household oximeters and popularizing in ordinary households.

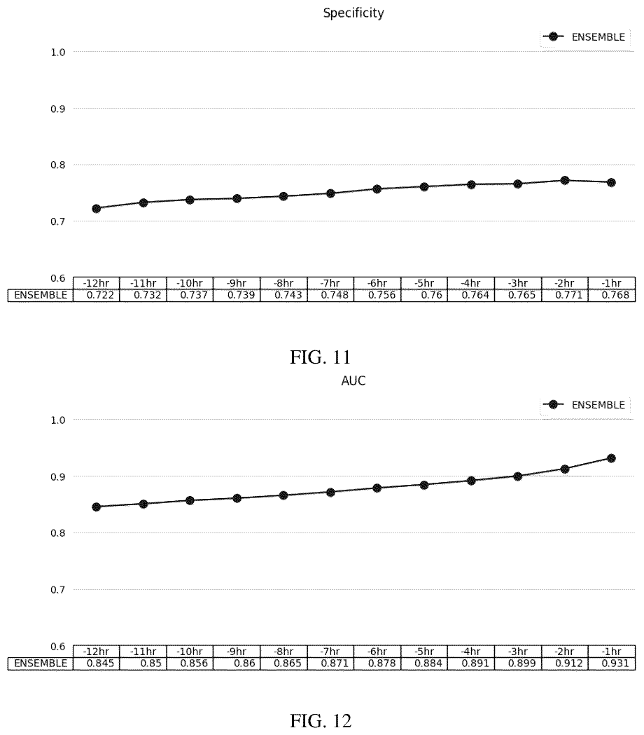

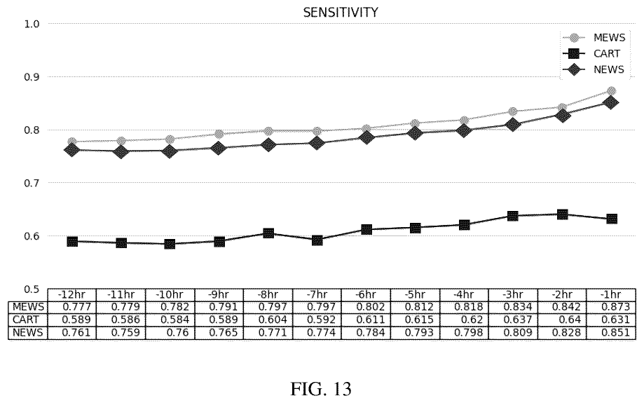

The test data of different time points were also analyzed to evaluate the model's prediction efficacy and to compare with other commonly used CA early warning systems. Physiological monitoring data of 23,457 surviving and 3,922 dead patients were used to predict the risk of cardiac arrest for consecutive 12 hours. Each prediction was performed by using 24 consecutive hours of heart rate (HR) and blood oxygen (SpO₂) measurements before the prediction time point. The results are as shown in FIG. 10, FIG. 11 and FIG. 12. The prediction efficacies of the Modified Early Warning Score (MEWS), the Cardiac Arrest Risk Triage (CART), and the National Early Warning Score (NEWS) are shown in FIG. 13 for comparison. From the perspective of sensitivity, at least from nine hours before CA occurrence, the early warning model of the present invention predicted 80% of sudden death in patients, and from one hour before CA occurrence the sensitivity even reached 90%. This performance is better than other three commonly used indicators (MEWS, CART and NEWS) which employ more physiological parameters (instead of 2 for the model of the present invention) in risk evaluation. From the perspective of specificity, more than 70% of non-sudden death patients were also successfully predicted. The AUC analysis also indicates a good prediction efficacy.

### Example 5

### Construction of a Real-time Blood Oxygen and Heart Rate Monitoring Platform

The AI prediction model trained in Example 1 is integrated into a blood oxygen and heart rate real-time monitoring platform to provide medical staff the prediction results of patients' conditions, especially the risk of cardiac arrest (CA). Referring to the configuration in FIG. 1, in this embodiment, the sensing module 110 uses Aulisa continuous wireless blood oxygen monitoring system to measure physiological data of the patient such as blood oxygen (SpO₂) and heart rate (HR). The sensing module 110 automatically and continuously measures and transmits the physiological data to the gateway 111 within a range of 10 meters through Bluetooth 4.0. After receiving the above physiological data, the gateway 111 transmits the measured physiological data to the database built in the server 120. The server 120 has the AI prediction model trained in Example 1, which can analyze the input physiological data of patient in real time, to monitor and predict the patient's risk index with zero time difference, so as to avoid sudden death (such as cardiac arrest) caused by silent hypoxia. In this example, the database is built using Microsoft SQL Server system, and Microsoft Power BI is used to present analysis data. The server 120 allows authorized remote devices 130 (such as remote computers) to access, so a medical staff can see the monitoring status and predictive analysis results of multiple patients at the same time, and the status of a patient can also be instantly share with multiple medical staff (such as physicians from different disciplines) for online consultation.

From the above results, it can be seen that the establishment and use of the blood oxygen system real-time monitoring and early warning platform can effectively reduce the risk of sudden death of patients with new coronary pneumonia due to quiet hypoxia, and the use of AI trained by machine learning can improve the ability of early warning analysis. Effective screening of high-risk cases that require special treatment and care can effectively reduce the burden of medical care and quickly provide appropriate medical treatment to patients when infectious diseases break out and the number of medical passports increases. And because the monitoring and early warning platform only needs to capture heart rate (HR) and blood oxygen (SpO₂) data to make predictions, it can be integrated into ordinary household blood oxygen machines, which is more suitable for ordinary household users.

In addition to the above physiological data, the real-time monitoring and early warning platform of the present invention can also combine other data that can be provided by medical staff or patients, such as height, weight, gender, age and other characteristics, combined with heart rate and blood oxygen data, so that the AI model can more accurately predict the CA risk index of the patient.

Although the invention has been disclosed by preferred embodiments, it is not intended to limit the invention. The skilled artisan may make modifications without departing from the spirit and scope of the invention. Thus, the protection scope of the present invention should be defined by the claims appended below.

### REFERENCE SIGNS

100 Blood oxygen and heart rate real-time monitory platform

110 Sensory module

111 Gateway

120 Server

130 Remote Devices

What is claimed is:

1. A machine learning model training method to train a sudden death prediction model for predicting the probability of sudden death, comprising using continuous physiological monitoring data and survival results of patients in a database for model training, wherein:
the physiological monitoring data consist essentially of heart rate (HR) and
blood oxygen (SpO₂) data; and
the survival results comprise categories of the survival status of the patients.

2. The method of claim 1, wherein the physiological monitoring data comprise continuous 24-hour physiological data of patients.

3. The method of claim 1, wherein the categories of the survival status comprise a category representing patient alive and a category representing patient death.

4. The method of claim 1, comprising performing synthetic minority oversampling technique (SMOTE) for categories with smaller data amount for repeated sampling, so that the amount of data of different categories are approximately equal.

5. The method of claim 1, wherein the machine learning model is trained with a long short-term memory recurrent neural network (LSTM-RNN).

6. The method of claim 5, wherein the LSTM-RNN employs two LSTM layers plus two fully connected layers after expansion of the LSTM layers, and then applies a Softmax layer to the last layer for training.

7. The method of claim 1, wherein the machine learning model is trained by temporal convolutional network (TCN).

8. A trained sudden death prediction model for predicting the probability of sudden death of a subject, the sudden death prediction model can use continuous physiological monitoring data of the subject to predict the health status of the subject, wherein:
the sudden death prediction model is a machine learning model; and
the physiological monitoring data consist essentially of heart rate (HR) and
blood oxygen (SpO₂) data.

9. The sudden death prediction model of claim 8, wherein the health status is a predicted survival status of the subject.

10. The sudden death prediction model of claim 8, wherein the health status is the probability of sudden death of the subject.

11. The sudden death prediction model of claim 8, wherein the model can predict sudden death of the subject 6 hours before occurrence of cardiac arrest.

12. A real-time monitoring system for monitoring and warning the risk of sudden death of patients, comprising at least one sensing module capable of real-time measurement and transmission of physiological data, a server, and a plurality of remote devices, wherein:
the sensing module is connected to the server, and the physiological data measured by the sensing module can be transmitted to the server in real time,
the physiological data comprise at least heart rate (HR) and blood oxygen (SpO₂) data of a subject;
the server comprises a prediction platform comprising a trained sudden death prediction model, and the trained sudden death prediction model is capable of predicting the health status of the subject only relying on the heart rate (HR) and blood oxygen (SpO₂) data of the subject; and
the server is connected with the remote devices, and can transmit the real-time physiological data measured by the sensing module and the health status predicted by the prediction platform to the remote devices with access authority.

13. The real-time monitoring system of claim 12, wherein the sensing module is a household pulse oximeter capable of collecting heart rate (HR) and blood oxygen (SpO₂) data.

14. The real-time monitoring system of claim 12, wherein the server comprises a SQL database system.

15. The real-time monitoring system of claim 12, wherein the server comprises a Power BI analysis reporting system.
